(19) United States
(12) Patent Application Publication
(10) Pub. No.: US 2026/0331295 A1
(43) Pub. Date: Oct. 22, 2026

(54) SYSTEM FOR HEALTH MANAGEMENT AND OPERATING METHOD THEREOF

(71) Applicant: ODN Co., Ltd., Seoul (KR)

(72) Inventors: Sang Youl RHEE, Seoul (KR); Hyun Jung PARK, Seoul (KR); Tae Hyun PARK, Seoul (KR); Seung Cheol AHN, Seoul (KR)

(21) Appl. No.: 19/648,550

(22) Filed: Apr. 15, 2026

(30) Foreign Application Priority Data
Apr. 17, 2025 (KR) 10-2025-0050273

(51) Int. Cl. G16H 50/30; G16H 10/60; G16H 15/00

(52) U.S. Cl. CPC G16H 50/30; G16H 10/60; G16H 15/00

(57) ABSTRACT

Provided are a system for health management and an operating method thereof. A health management system may include a Backend For Frontend (BFF) server configured to perform an interface function with a health management app installed in a terminal of a user, and receive a service request from the health management app; a Fast Healthcare Interoperability Resource (FHIR) relay server configured to generate an FHIR request by converting the service request according to an FHIR protocol, and transmit the FHIR request to an internal network of a medical institution; and an FHIR server located in the internal network and configured to perform processing for the FHIR request. Here, the system may interoperate with a legacy system of the medical institution, and may perform processing based on health data of the user independently of the legacy system.

### CROSS-REFERENCE TO RELATED APPLICATION

This application claims priority to Korean Patent Application No. 10-2025-0050273, filed on Apr. 17, 2025, in the Ministry of Intellectual Property (MOIP) of the Republic of Korea, the disclosure of which is incorporated herein by reference.

### STATEMENT REGARDING SPONSORED RESEARCH OR DEVELOPMENT

The present disclosure was made with the support of the Ministry of Trade, Industry and Energy (MOTIE) of the Republic of Korea, under a program supervised by the Korea Institute for Advancement of Technology (KIAT), under Project Unique Number 2410013887 and Project Number 07892971, entitled “Industrial Technology Development Program for the Bio Industry (R&D),” specifically the project titled “Development of an AI-based Biosignal Monitoring and Real-time Early Warning System for Severe Trauma Patients, Beacon (Biosignal Emergency Advanced Critical Observation Network),” performed by ODN Co., Ltd. during the period from Jan. 1, 2026 to Dec. 31, 2026.

### BACKGROUND

### Field of the Disclosure

The present disclosure relates to a technology for monitoring and managing a health of a user.

### Description of the Related Art

Recently, various types of health management apps that collect physical information or health habit information of a user and manage a health state based thereon are being released with the development of wearable devices and mobile healthcare technologies. Such health management apps perform functions such as providing personalized feedback based on user input regarding weight, diet, sleep, and exercise, or visualizing the user's health state by utilizing this information.

However, since most conventional health management apps have a structure that is not linked with a legacy system of a medical institution, medical information (e.g., prescription information, blood test results, etc.) of the user managed by the medical institution cannot be integratedly checked within the app, and health information of the user collected through the health management app is also difficult to be utilized in the medical institution. Therefore, existing health management apps have a clear limitation in providing a comprehensive and integrated health management service to the user.

### SUMMARY

Therefore, the present disclosure has been made in view of the above problems, and it is an object of the present disclosure to provide a system capable of providing a comprehensive and integrated health management service to a user, and an operating method thereof.

Specifically, a technical problem to be solved by some embodiments of the present disclosure is to provide a structure of a health management system capable of being effectively linked with a legacy system of a medical institution, and an operating method thereof.

It is another object of the present disclosure to provide a system capable of early predicting an occurrence of a health abnormality based on health data of the user, and supporting a preemptive management measure, and an operating method thereof.

The technical problems of the present disclosure are not limited to the above-mentioned technical problems, and other technical problems not mentioned will be clearly understood by those skilled in the art to which the present disclosure pertains from the following description.

In accordance with an aspect of the present disclosure, the above and other objects can be accomplished by the provision of a system for health management, including: a Backend For Frontend (BFF) server configured to perform an interface function with a health management app installed in a terminal of a user, and receive a service request from the health management app; a Fast Healthcare Interoperability Resource (FHIR) relay server configured to generate an FHIR request by converting the service request according to an FHIR protocol, and transmit the FHIR request to an internal network of a medical institution; and an FHIR server located in the internal network and configured to perform processing for the FHIR request. Here, the system may interoperate with a legacy system of the medical institution, and may perform processing based on health data of the user independently of the legacy system.

In some embodiments, the legacy system may include an Electronic Medical Record (EMR) system.

In some embodiments, the system may provide at least a part of the health data to the legacy system, and may be provided with medical information of the user from the legacy system.

In some embodiments, the FHIR relay server may transmit the FHIR request to the FHIR server through a gateway server that controls access to the internal network.

In some embodiments, the service request may include a verification request on whether the user is a registered patient in the medical institution, the verification request may include personal identification information of the user and may be performed according to a membership registration application through the health management app, the FHIR request may include an FHIR verification request corresponding to the verification request, and the FHIR server may check whether the user is a registered patient through the legacy system using the personal identification information, and may return an FHIR response including a verification result to the FHIR relay server.

In some embodiments, the service request may include an inquiry request for prescription information, the inquiry request may include patient identification information registered in the medical institution, the FHIR request may include an FHIR inquiry request corresponding to the inquiry request, and the FHIR server may acquire the prescription information through the legacy system using the patient identification information, and return an FHIR response including the prescription information to the FHIR relay server.

In some embodiments, the service request may include a submission request for a health record, the FHIR request may include an FHIR submission request corresponding to the submission request, and the FHIR server may store the health record in a database accessible by the legacy system, and return an FHIR response including the storage result to the FHIR relay server.

In some embodiments, the system may further include an internal integration server providing the health data by visualizing the health data through a dashboard, wherein the health data includes body composition analysis information of the user and a health record submitted by the user through the health management app, wherein the body composition analysis information is acquired from a body composition analysis server included in the legacy system, and the dashboard is configured to be accessible through a User Interface (UI) of an Electronic Medical Record (EMR) system included in the legacy system.

In some embodiments, the health record may include health habit information and weight information, and the dashboard may include: a first interface displaying basic information of the user; a second interface displaying the health habit information; and a third interface displaying physical information including the weight information and the body composition analysis information, wherein the second interface further displays an evaluation score calculated based on the health habit information.

In some embodiments, the internal integration server may predict a success rate for a health target of the user by analyzing the health record, and may further provide the predicted success rate through the dashboard.

In some embodiments, the internal integration server may predict the success rate through a machine learning model, the health record may include health habit information and weight information, and the machine learning model may include: a habit encoder configured to generate a habit embedding by encoding the health habit information; a willingness encoder configured to generate a willingness embedding by encoding willingness information of the user, the willingness information including at least one of a weight change of the user, a voluntary submission frequency for the health habit information, and a change frequency of the health target; an environment encoder configured to generate an environment embedding by encoding living environment information of the user; an aggregator configured to generate an integrated embedding by aggregating the habit embedding, the willingness embedding, and the environment embedding; and a predictor configured to predict the success rate based on the integrated embedding.

In some embodiments, the system may further include an internal integration server configured to predict a risk level of an occurrence of a health abnormality for the user by analyzing the health data through a machine learning model, wherein the machine learning model includes: an encoder configured to generate variable embeddings corresponding to predefined health variables; an interaction encoder configured to generate interaction embeddings corresponding to predefined health variable pairs based on the variable embeddings; a variable weight estimator configured to estimate a variable weight specialized for the user with respect to each of the health variables based on clinical information of the user acquired from the legacy system; an interaction weight estimator configured to estimate an interaction weight specialized for the user with respect to each of the health variable pairs based on the clinical information; an aggregator configured to generate an integrated embedding by aggregating the variable embeddings, to which the variable weight is reflected, and the interaction embeddings to which the interaction weight is reflected; and a predictor configured to predict the risk level based on the integrated embedding.

In some embodiments, the internal integration server may: provide a notification related to the health abnormality when the predicted risk level is a threshold value or more and a preset standby timer expires, immediately provide the notification when an event increasing a risk of the occurrence of the health abnormality for the user is detected before the standby timer expires, and reset the standby timer and predicts the risk level again through the machine learning model when an event decreasing the risk of the occurrence of the health abnormality for the user is detected before the standby timer expires.

### BRIEF DESCRIPTION OF THE DRAWINGS

The above and other objects, features and other advantages of the present disclosure will be more clearly understood from the following detailed description taken in conjunction with the accompanying drawings, in which:

FIG. 1 is a view for explaining an exemplary operating environment of a health management system according to some embodiments of the present disclosure;

FIG. 2 is an exemplary view for explaining an internal structure and operation of the health management system according to some embodiments of the present disclosure;

FIGS. 3 to 5 are exemplary flowcharts illustrating an operating method of the health management system according to some embodiments of the present disclosure;

FIG. 6 is an exemplary view for explaining a screen configuration of a dashboard according to some embodiments of the present disclosure;

FIGS. 7A to 7D illustrate implementation examples of the dashboard;

FIG. 8 is an exemplary view illustrating input/output data of a machine learning model predicting health abnormality information according to some embodiments of the present disclosure;

FIG. 9 is an exemplary view for explaining a structure and operation of the machine learning model according to some embodiments of the present disclosure;

FIG. 10 is an exemplary view for explaining a model training method based on multi-task learning according to some embodiments of the present disclosure;

FIG. 11 is an exemplary view for explaining a structure and operation of a machine learning model predicting a success rate for a health target according to some embodiments of the present disclosure; and

FIG. 12 illustrates an exemplary computing device capable of implementing the health management system and/or components thereof according to some embodiments of the present disclosure.

### DETAILED DESCRIPTION OF THE DISCLOSURE

Hereinafter, various embodiments of the present disclosure will be described in detail with reference to the accompanying drawings. Advantages and features of the present disclosure and methods of achieving them will become apparent with reference to embodiments described below in detail in conjunction with the accompanying drawings. However, the technical spirit of the present disclosure is not limited to the following embodiments, but may be implemented in various different forms. The following embodiments are merely provided to make the technical spirit of the present disclosure complete and to fully inform those of ordinary skill in the art to which the present disclosure pertains of the scope of the present disclosure, and the technical spirit of the present disclosure is only defined by the scope of the claims.

When it is determined that a detailed description of a related known configuration or function may obscure the gist of the present disclosure in describing the various embodiments of the present disclosure, the detailed description thereof will be omitted.

Unless otherwise defined, terms (including technical and scientific terms) used in the following embodiments may be used with meanings commonly understood by those of ordinary skill in the art to which the present disclosure pertains, but this may vary depending on the intentions of technicians working in the related field, precedents, the emergence of new technologies, and the like. The terms used in the present disclosure are for describing the embodiments and are not intended to limit the scope of the present disclosure.

Singular expressions used in the following embodiments include plural concepts unless the context clearly specifies them as singular. In addition, the plural expressions include singular concepts unless the context clearly specifies them as plural.

In addition, terms such as first, second, A, B, (a), and (b) used in the following embodiments are merely used to distinguish one component from another component, and the essence, order, or sequence of the corresponding component is not limited by the terms.

Components described using terms such as “~unit”, “module”, “block”, and “~er” or “~or” in the following embodiments and functional blocks shown in the drawings may be implemented in the form of software, hardware, or a combination thereof. The software may include, for example, machine code, firmware, embedded code (or software), application software, or a combination thereof. In addition, the hardware may include, for example, an electrical circuit, an electronic circuit, a processor, a computer, an integrated circuit, integrated circuit cores, a passive element, or a combination thereof. As a more specific example, the “~unit”, the “module”, and the like may include components (such as software components, object-oriented software components, class components, and task components), processes, functions, properties, procedures, subroutines, segments of program code, drivers, firmware, microcode, circuitry, data, databases, data structures, tables, arrays, and variables.

Hereinafter, various embodiments of the present disclosure will be described in detail with reference to the accompanying drawings.

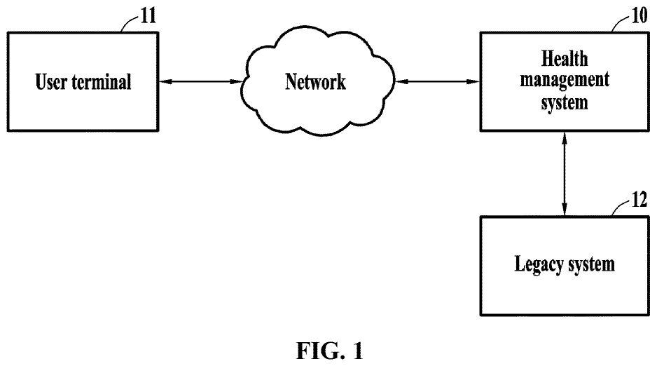

FIG. 1 is a view for explaining an exemplary operating environment of a health management system 10 according to some embodiments of the present disclosure.

As shown in FIG. 1, the health management system 10 according to embodiments is a computing device/system equipped with a health management function for at least one user, and may interoperate with a user terminal 11 and a legacy system 12 of a medical institution. However, the scope of the present disclosure is not limited thereto. The health management system 10 may be linked with a plurality of user terminals to support health management for a plurality of users.

In the operating environment illustrated in FIG. 1, the health management system 10 may collect health data (e.g., data on health habits, weight, etc.) of the user through a health management app installed in the user terminal 11, and perform a health management function (e.g., notification of a health abnormality, etc.) for the user by analyzing the collected health data.

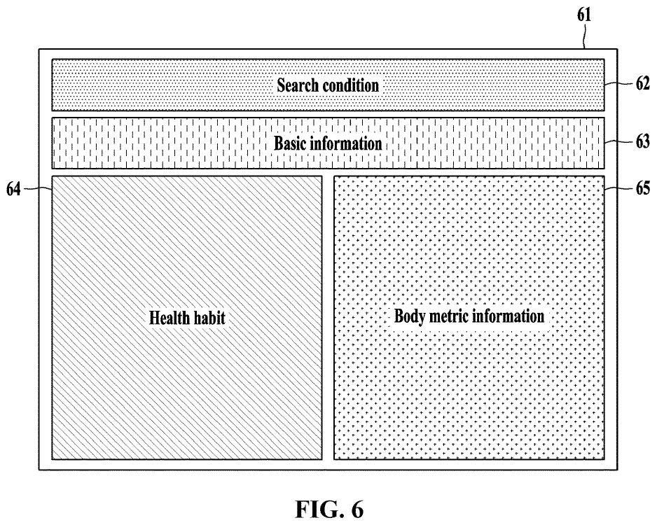

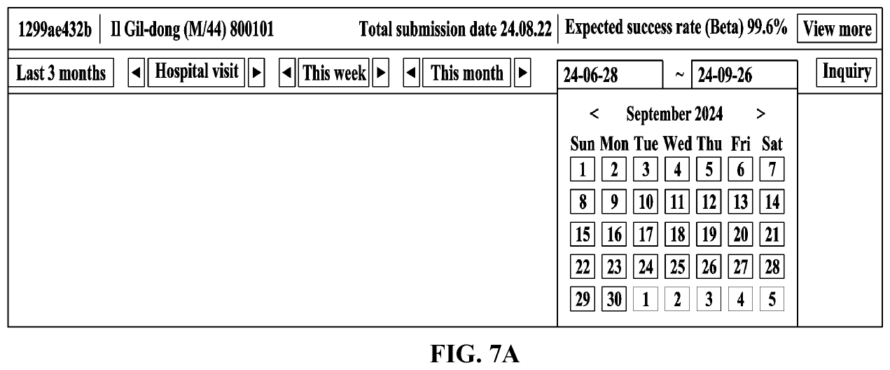

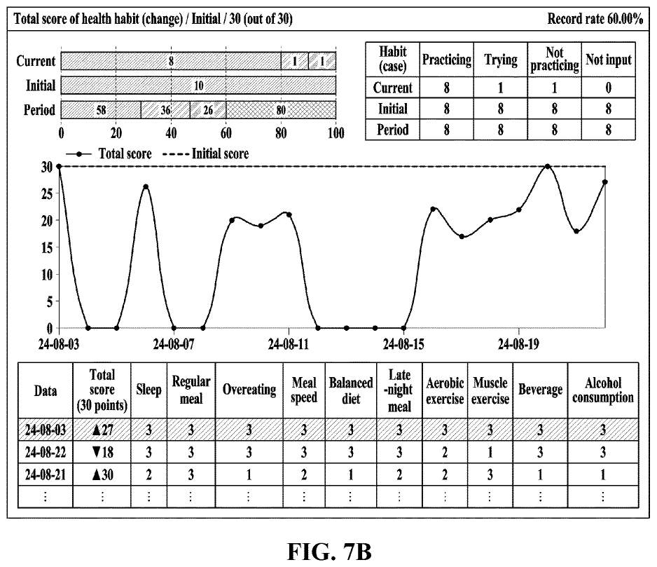

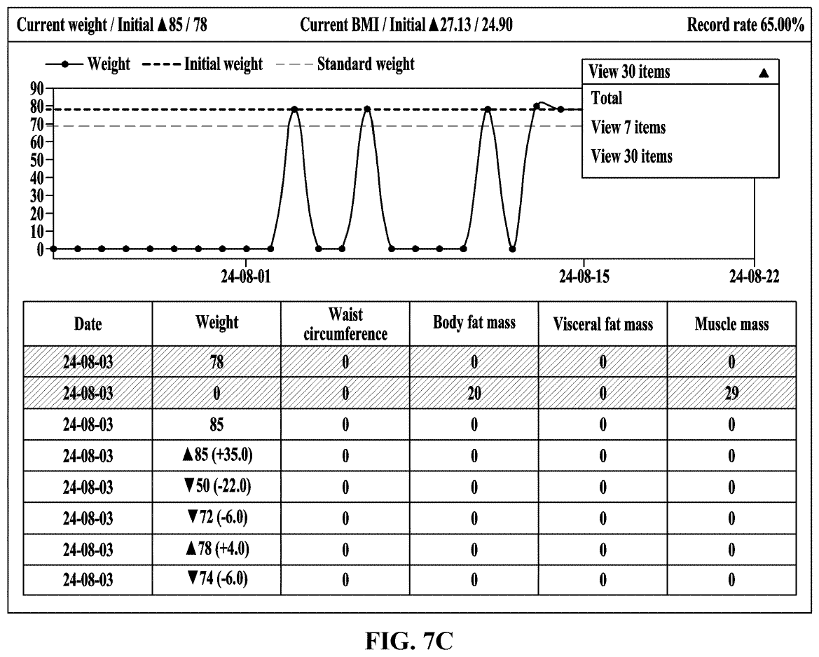

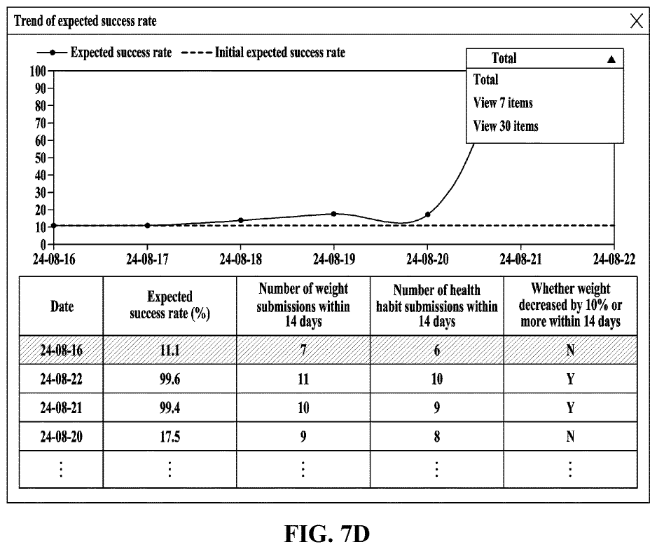

In addition, the health management system 10 may provide health-related information (e.g., the health data and/or an analysis result thereof) of the user to the legacy system 12 of the medical institution (i.e., may share the health-related information with the medical institution). In this case, since the health management system 10 performs processing based on the health data independently of the legacy system 12, the health-related information of the user may be reflected (or integrated) into the legacy system 12 without affecting a structure or performance of the legacy system 12. The health management system 10 may visualize and provide the health-related information of the user through a dashboard, which will be described with reference to FIGS. 6 to 7D and the like.

In addition, the health management system 10 may perform a customized health management for the user by acquiring medical information (e.g., various clinical information) of the user from the legacy system 12 and jointly analyzing the health data and the medical information of the user. For example, the health management system 10 may make an early prediction of a risk level of an occurrence of a health abnormality within a specific time (e.g., 24 hours, 6 hours, etc.) through a machine learning model, and preemptively provide a notification for health management to the user based thereon. Such a notification may be provided through, for example, a health management app installed in the user terminal 11, and may also be provided to medical staff (e.g., when the user is a patient registered in a medical institution, the notification may also be provided to medical staff in charge). As another example, the health management system 10 may detect the health abnormality in real time or near real time through the machine learning model.

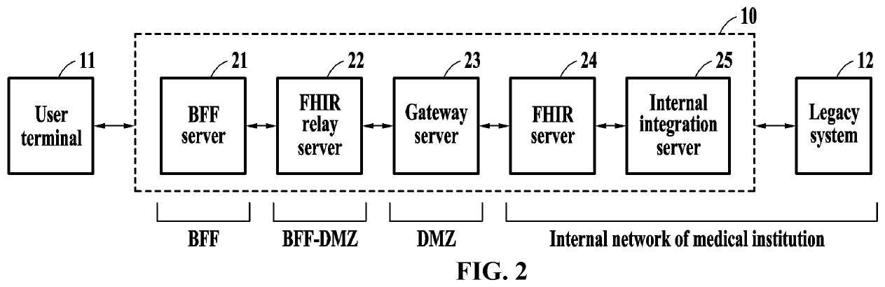

A more detailed description on an internal structure and operation of the health management system 10 will be described below with reference to FIG. 2 and subsequent drawings.

In some cases, the above-described health management system 10 may be named a ‘health management platform’, a ‘healthcare system/platform’, an ‘integrated health management service system/platform’, or the like.

The user terminal 11 refers to a device on a side of the user (i.e., a computing device) capable of interoperating with the health management system 10 and the like. A health management app interoperating with the health management system 10 may be installed in the user terminal 11, and the user may be provided with a health management function through the corresponding app (e.g., submitting a health record, receiving a health abnormality notification, etc.).

The user terminal 11 may be implemented as various types of devices such as a smartphone, a tablet, a desktop, a smartwatch, or other wearable devices, for example, but is not limited thereto.

The legacy system 12 refers to an information system already built and operating in the medical institution. The legacy system 12 may acquire health-related information (e.g., health data, an analysis result, etc.) of the user from the health management system 10, and may provide the medical information of the user to the health management system 10.

The medical information may include, for example, clinical information and non-clinical information. In addition, the clinical information may include, for example, a type of patient (e.g., pre-surgery, post-surgery, emergency patient, inpatient, etc.), demographic information, diagnosis information (e.g., a diagnosis name, etc.), prescription information, a test result (e.g., a blood test result, a physical test result), surgery information, a medication history, a disease history, body composition analysis information (e.g., information measured by a Dual Energy X-ray Absorptiometry (DEXA) technique, a Bioelectrical Impedance Analysis (BIA) technique), and the like, but is not limited thereto. In addition, the non-clinical information may include, for example, administrative information, insurance information, and the like, but is not limited thereto.

Examples of the legacy system 12 may include an Electronic Medical Record (EMR), a Hospital Information System (HIS), an Order Communication System (OCS), a Picture Archiving and Communication System (PACS), a Laboratory Information System (LIS), and the like, but are not limited thereto. For example, a scope of the legacy system 12 may also include a body composition analysis server located in an internal network of the medical institution. Since the exemplified systems are generally built based on a closed structure or a non-standard interface, direct interoperation with an external system is often limited.

As illustrated in FIG. 1, the user terminal 11, the health management system 10 and the like may communicate through a network. Here, the network may be implemented as all kinds of wired/wireless networks such as a Local Area Network (LAN), a Wide Area Network (WAN), a mobile radio communication network, a Wireless Broadband Internet (Wibro), and the like.

As described above, the exemplary operating environment of the health management system 10 according to some embodiments of the present disclosure has been described with reference to FIG. 1. Hereinafter, an internal structure and an operation of the above-described health management system 10 will be described with reference to FIG. 2.

FIG. 2 is an exemplary view for explaining the internal structure and operation of the health management system 10 according to some embodiments of the present disclosure.

As shown in FIG. 2, in some embodiments, the health management system 10 may include a Backend For Frontend (BFF) server 21, an FHIR relay server 22, a gateway server 23, an FHIR server 24, and an internal integration server 25. Here, the FHIR server 24, the internal integration server 25, and the legacy system 12 may be located in an internal network of the medical institution, and the gateway server 23 may be disposed in a Demilitarized Zone (DMZ) area for security of the internal network. When configured in this way, an interoperation between the health management system 10 and the legacy system 12 may be effectively performed. Hereinafter, an operation of each server will be described.

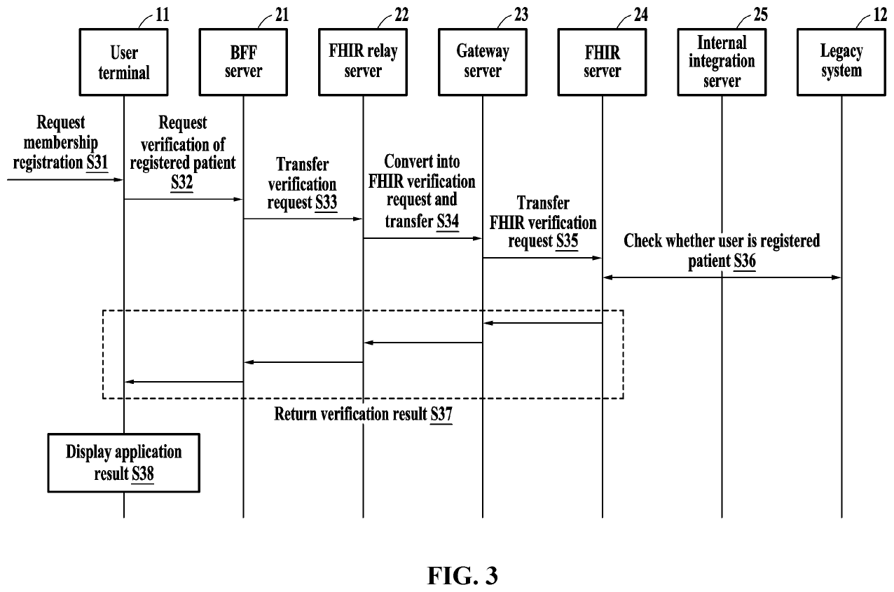

The BFF server 21 is a server responsible for an interface function with the health management app installed in the user terminal 11. For example, the BFF server 21 may receive a service request (e.g., an API request) of the health management app, and return (or transmit/transfer, etc.) a service response including a processing result thereof to the health management app. Such a service request may be performed by, for example, an API call method, and in this case, the BFF server 21 may be understood to correspond to an API server. However, the scope of the present disclosure is not limited thereto. Specific examples of the service request will be described with reference to FIGS. 3 to 5. The BFF server 21 may transfer (or transmit) a specific service request to the FHIR relay server 22 to process the corresponding request.

Next, the FHIR relay server 22 is a server performing processing according to an FHIR protocol. Specifically, the FHIR relay server 22 may receive a service request from the BFF server 21 and generate an FHIR request (e.g., an API request) by converting it according to the FHIR protocol. Then, the FHIR relay server 22 may transmit the FHIR request to the FHIR server 24 of the internal network through the gateway server 23, and receive a response (i.e., an FHIR response) to the FHIR request from the FHIR server 24. Then, the FHIR relay server 22 may transfer (or return) the FHIR response to the BFF server 21 by converting it into a format of a service response readable by the BFF server 21. The FHIR relay server 22 may be understood to serve as an internal FHIR client.

Next, the gateway server 23 is a server responsible for a gateway role of relaying communication with the outside while maintaining security of the internal network. That is, the gateway server 23 may perform an access control to the internal network of the medical institution, and transfer (or transmit) the FHIR request, received from the FHIR relay server 22, to the FHIR server 24. In addition, the gateway server 23 may transfer (or return) the FHIR response received from the FHIR server 24 to the FHIR relay server 22.

Next, the FHIR server 24 is a server reading and processing the FHIR request. Specifically, the FHIR server 24 may read and process the FHIR request received from the gateway server 23, and generate and return (or transmit) an FHIR response thereto to the gateway server 23. In some cases, the FHIR server 24 may provide (or may share) a health record of the user included in the FHIR request to the internal integration server 25 and/or the legacy system 12. Here, providing the health record to a specific server and/or system may encompass not only a concept of transmitting the corresponding health record, but also a concept of storing the corresponding health record in a database (i.e., a storage) accessible by the corresponding server and/or system.

Finally, the internal integration server 25 is a server integratingly analyzing and managing health data, medical information/data, and the like of the user. The internal integration server 25 may acquire, for example, medical information of the user from the legacy system 12, and may acquire health data of the user from the FHIR server 24 (e.g., acquire the health data from a database of the FHIR server 24). The internal integration server 25 may integrally manage health data, medical data and the like of a plurality of users.

In addition, the internal integration server 25 may provide health-related information (e.g., health data, an analysis result, etc.) of the user to the user and/or medical staff through a dashboard. For example, the internal integration server 25 may visually provide the health data of the user to the medical staff using the legacy system 12 (e.g., an EMR system), which will be described with reference to FIGS. 6 to 7D. The internal integration server 25 may integrally provide health-related information of a plurality of users through the dashboard.

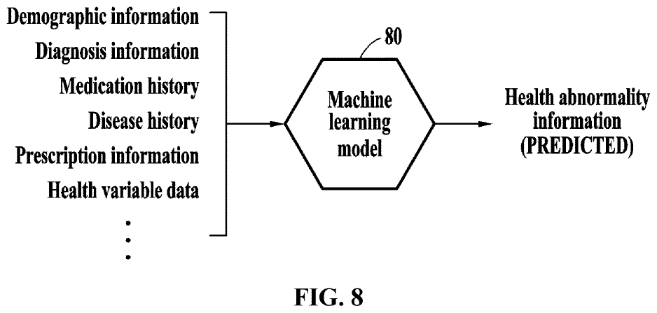

In addition, the internal integration server 25 may make an early prediction of health abnormality information of the user through a machine learning model, and provide a notification to the user and/or the medical staff based on a prediction result. This will be described with reference to descriptions of FIGS. 8 to 10 and the like.

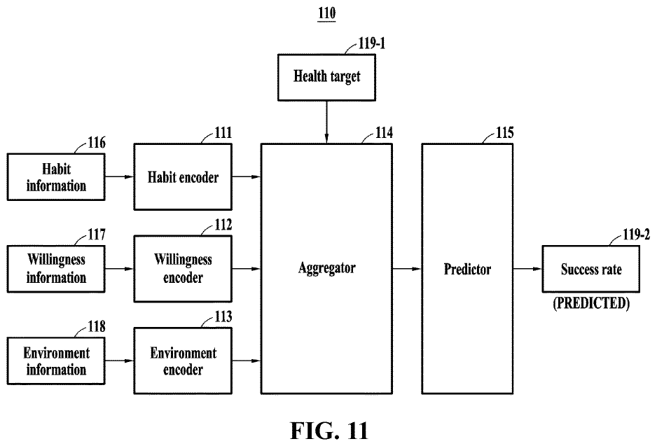

In addition, the internal integration server 25 may predict a success rate (i.e., an expected success rate) of the user for a health target through the machine learning model, which will be described with reference to a description of FIG. 11 and the like.

The above-described health management system 10 or components 21 to 25 thereof may be implemented as at least one computing device. For example, all functions of the internal integration server 25 may be implemented in one computing device, and a first function of the internal integration server 25 may be implemented in a first computing device and a second function thereof may be implemented in a second computing device. Alternatively, a specific function of the internal integration server 25 may be implemented in a plurality of computing devices.

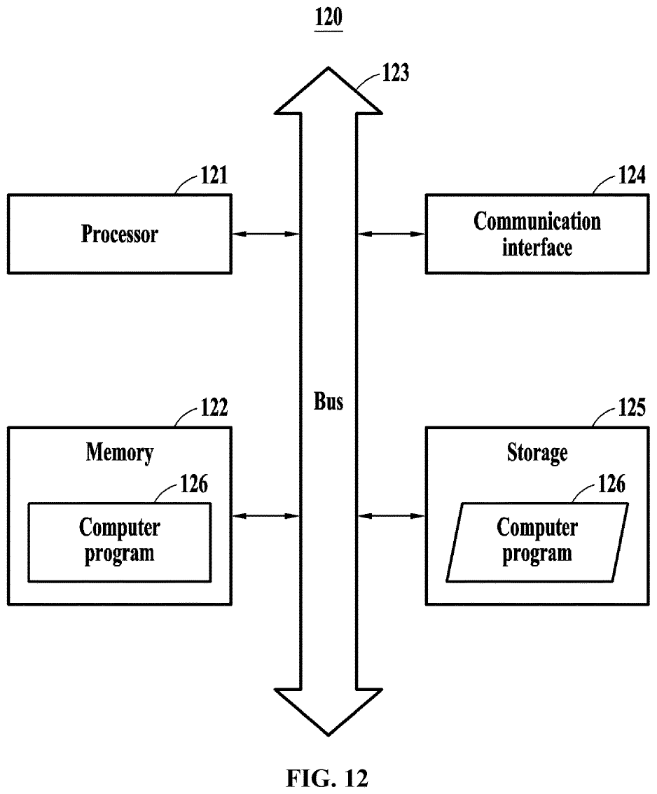

The computing device may encompass any device having a computing (or processing) function, and an example of such a device will be described with reference to FIG. 12. Since the computing device is an aggregate in which various components (e.g., a memory, a processor, etc.) interact with each other, it may be named a ‘computing system’ in some cases. Of course, the term “computing system” may also encompass a concept of an aggregate in which a plurality of computing devices interact with each other.

As described above, the internal structure and operation of the health management system 10 have been described with reference to FIG. 2. Hereinafter, a specific operating method of the above-described health management system 10 will be described with reference to FIG. 3 and subsequent drawings.

Hereinafter, for convenience of understanding, the description will be continued assuming a case in which the health management system 10 is configured as shown in FIG. 2 in the environment illustrated in FIG. 1. In addition, for convenience of description, the health management system 10 will be abbreviated as a ‘system’.

The operating method of the system 10 may be performed in relation to, for example, a membership registration process, a prescription information inquiry process, a health record submission process, and the like. Hereinafter, the operating method of the health management system 10 related to each process will be described in detail.

FIG. 3 is an exemplary flowchart illustrating a membership registration process according to some embodiments of the present disclosure.

As illustrated in FIG. 3, the process relates to a procedure in which a user registers as a member of the health management system 10 (or a health management service) through a user terminal 11.

As illustrated, the process may begin at step S31, in which the user terminal 11 receives a membership registration application. For example, the user may apply for membership registration through a health management app of the user terminal 11 and input personal identification information (e.g., name, date of birth, gender, mobile phone number, etc.).

Steps S32 to S37, which will be described below, may be understood as procedures performed to provide an integrated health management service through linkage with a medical institution when the user is a patient registered in the medical institution.

In step S32, the user terminal 11 transmits a service request (hereinafter, abbreviated as a ‘verification request’) for verifying whether the corresponding user is a patient registered in the medical institution to the BFF server 21. For example, the health management app of the user terminal 11 may generate the verification request including the personal identification information of the corresponding user and transmit it to the BFF server 21.

In step S33, the BFF server 21 transfers the received verification request to the FHIR relay server 22.

In step S34, the FHIR relay server 22 converts the received verification request according to the FHIR protocol and transmits it to the gateway server 23. That is, the FHIR relay server 22 converts the corresponding verification request to generate an FHIR verification request, and transmits it to the FHIR server 24 of the internal network through the gateway server 23.

In step S35, the gateway server 23 transfers the FHIR verification request to the FHIR server 24.

In step S36, the FHIR server 24 processes the FHIR verification request by interoperating with the legacy system 12 (e.g., an EMR system) to verify whether the user is a registered patient. For example, the FHIR server 24 may look up a patient registration number, managed by the legacy system 12, using the personal identification information included in the corresponding FHIR verification request. Such a process may be performed through an API provided by the legacy system 12, but the scope of the present disclosure is not limited thereto.

In step S37, the FHIR server 24 returns the verification result. Specifically, the FHIR server 24 may generate and return an FHIR response including the verification result (e.g., whether the user is a registered patient, the patient registration number, etc.). The FHIR response may be transmitted to the FHIR relay server 22 via the gateway server 23, and may be converted into a format of a service response readable by the BFF server 21 in the FHIR relay server 22 to be transferred to the BFF server 21. Then, the BFF server 21 may transfer it to the user terminal 11.

In step S38, the user terminal 11 displays (or provides) a result of the membership registration application (e.g., success, failure, the patient registration number, etc.) to the user.

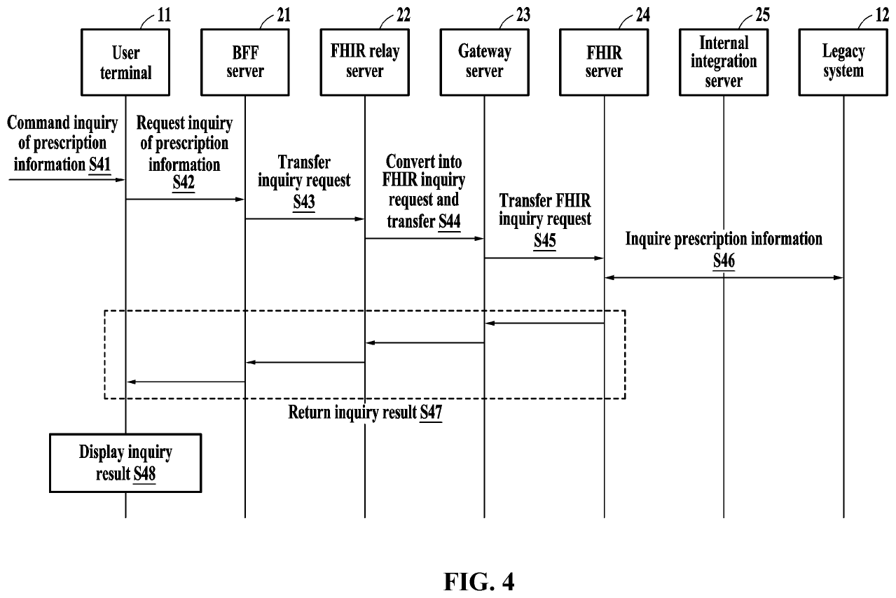

FIG. 4 is an exemplary flowchart illustrating a prescription information inquiry process according to some embodiments of the present disclosure.

As shown in FIG. 4, the process relates to a procedure in which a user inquires about prescription information managed in a medical institution through the user terminal 11, and may be performed when the user is a patient registered in the medical institution.

As illustrated, the process may begin at step S41, in which the user terminal 11 receives a prescription information inquiry request. For example, the user may request to inquire about the prescription information through the health management app of the user terminal 11 (e.g., by clicking an inquiry button, etc.).

In step S42, the user terminal 11 transmits a service request (hereinafter, abbreviated as an ‘inquiry request’) for inquiring about the prescription information to the BFF server 21. For example, the health management app of the user terminal 11 may generate the inquiry request including patient identification information (e.g., a patient registration number, etc.) of the corresponding user, and transmit it to the BFF server 21.

In step S43, the BFF server 21 transfers the received inquiry request to the FHIR relay server 22.

In step S44, the FHIR relay server 22 converts the received inquiry request according to the FHIR protocol, and transmits it to a gateway server 24. That is, the FHIR relay server 22 converts the corresponding inquiry request to generate an FHIR inquiry request, and transmits it to the FHIR server 24 of the internal network through the gateway server 24.

In step S45, the gateway server 24 transfers the FHIR inquiry request to the FHIR server 24.

In step S46, the FHIR server 24 processes the FHIR inquiry request by interoperating with the legacy system 12 (e.g., an EMR system) to inquire about the prescription information of the user. For example, the FHIR server 24 may inquire about the prescription information of the user managed in the legacy system 12 by using the patient identification information included in the corresponding FHIR inquiry request. Such a process may be performed through an API provided by the legacy system 12, but the scope of the present disclosure is not limited thereto.

In step S47, the FHIR server 24 returns an inquiry result. Specifically, the FHIR server 24 may generate and return an FHIR response including the inquiry result (e.g., success, failure, the inquired prescription information, etc.). The FHIR response may be transmitted to the FHIR relay server 22 via the gateway server 24, and may be converted into a service response readable by the BFF server 21 in the FHIR relay server 22 to be transferred to the BFF server 21. Then, the BFF server 21 may transfer it to the user terminal 11.

In step S48, the user terminal 11 displays (or provides) the prescription information inquiry result to the user.

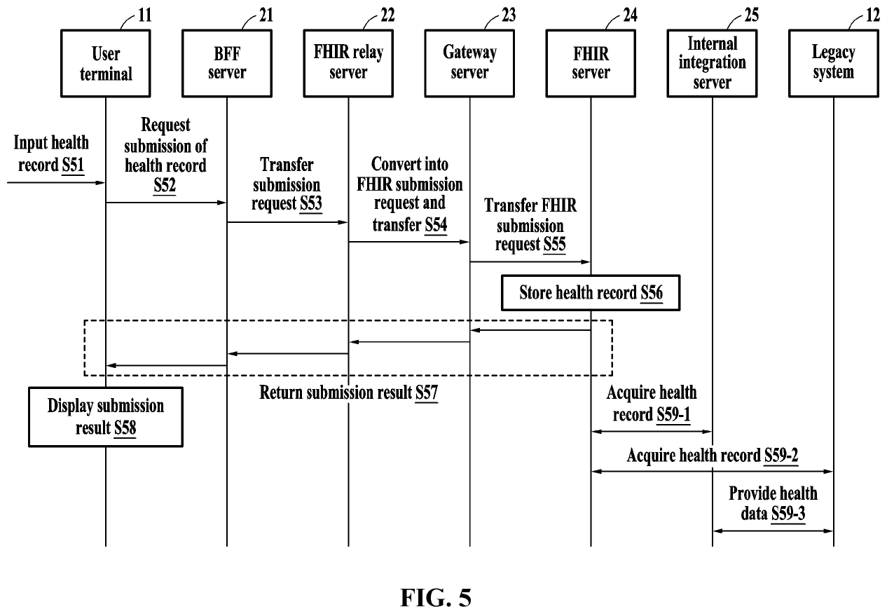

FIG. 5 is an exemplary flowchart illustrating a health record submission process according to some embodiments of the present disclosure.

As shown in FIG. 5, the process relates to a procedure in which a user submits a health record through the user terminal 11 to be provided with a health management service. The health record may include, for example, but is not limited to, information on health habits (e.g., habits related to exercise, meal, sleep, etc.) practiced (and/or not practiced) by the user, weight information, and the like.

As illustrated, the process may begin at step S51, in which the user terminal 11 receives an input of a health record. For example, the user may input a health record through the health management app of the user terminal 11 and request submission thereof (e.g., by inputting the health record and clicking a submission button, etc.).

In step S52, the user terminal 11 transmits a service request (hereinafter, abbreviated as a ‘submission request’) for submitting the health record to the BFF server 21. For example, the health management app of the user terminal 11 may generate the submission request including the inputted health record and transmit it to the BFF server 21.

In step S53, the BFF server 21 transfers the received submission request to the FHIR relay server 22.

In step S54, the FHIR relay server 22 converts the received submission request according to the FHIR protocol and transmits it to the gateway server 23. That is, the FHIR relay server 22 converts the submission request to generate an FHIR submission request, and transmits it to the FHIR server 24 of the internal network through the gateway server 23.

In step S55, the gateway server 23 transfers the FHIR submission request to the FHIR server 24.

In step S56, the FHIR server 24 processes the FHIR submission request by storing the health record. For example, the FHIR server 24 may store the health record, included in the FHIR submission request, in a database accessible by the internal integration server 25. Alternatively, the FHIR server 24 may store the health record in the database and simultaneously transmit it to the internal integration server 25.

In step S57, the FHIR server 24 returns a submission result. Specifically, the FHIR server 24 may generate and return an FHIR response including the submission result (e.g., success, failure, etc.). The FHIR response may be transmitted to the FHIR relay server 22 via the gateway server 23, and may be converted into a service response of a format readable by the BFF server 21 in the FHIR relay server 22 to be transferred to the BFF server 21. Then, the BFF server 21 may transfer it to the user terminal 11.

In step S58, the user terminal 11 displays the health record submission result to the user.

Steps S59-1 to S59-3, which will be described below, may be understood as being performed as necessary and being performed regardless of an order.

In step S59-1, the internal integration server 25 acquires the health record of the user from the FHIR server 24. For example, the internal integration server 25 may periodically or aperiodically acquire the health record of the user from a storage of the FHIR server 24.

In step S59-2, the legacy system 12 acquires the health record of the user from the FHIR server 24. For example, the legacy system 12 may periodically or aperiodically acquire the health record of the user from the storage of the FHIR server 24.

In step S59-3, the internal integration server 25 provides health data (or health-related information including an analysis result thereof) for at least one user to the legacy system 12. Here, the health data may include, for example, the health record, body composition analysis information, and the like of the user, and the body composition analysis information may be acquired from a body composition analysis server included in the legacy system 12. However, the scope of the present disclosure is not limited thereto.

For example, the internal integration server 25 may provide a dashboard including the health data of the user in response to a request of the legacy system 12 (e.g., an EMR system). As a more specific example, when medical staff requests to view the health data of the user (i.e., a registered patient) on a User Interface (UI, i.e., a UI of an EMR client) of the EMR system (e.g., 12), the dashboard may be provided to the medical staff through the UI (i.e., the dashboard may be configured to be accessible through the UI of the EMR system). A screen configuration and the like of the dashboard will be described with reference to FIGS. 6 to 7D.

In some cases, the dashboard may be named a ‘monitoring interface’, a ‘monitoring screen’, a ‘monitoring board’, a ‘monitoring User Interface (UI)’, a ‘monitoring Graphical User Interface (GUI)’, or the like.

The dashboard may be designed in various forms, and a specific form thereof may vary depending on embodiments. A specific design example of the dashboard will be described below with reference to FIGS. 6 to 7D.

Meanwhile, the internal integration server 25 may perform various functions for health management of the user based on the health records of the user acquired (or collected) from the FHIR server 24 and the medical information acquired from the legacy system 12.

For example, the internal integration server 25 may detect a health abnormality (e.g., a rapid weight change, an occurrence of a disease, etc.) of the user in real time or near real time by analyzing the health records (e.g., a series of health records). The internal integration server 25 may detect such a health abnormality using a machine learning model, or may detect the health abnormality in another way.

As another example, the internal integration server 25 may make an early prediction of various information regarding the health abnormality by comprehensively analyzing health variable data and medical information (e.g., clinical information, etc.) of the user through the machine learning model. For example, the internal integration server 25 may predict a risk level (i.e., a probability) of an occurrence of the health abnormality within a specific time/period (e.g., 24 hours, etc.) from a current time point through the machine learning model, and may also predict an occurrence time point (i.e., an expected occurrence time point), type, occurrence cause, duration, and the like of the health abnormality. This example will be described in detail below with reference to FIGS. 8 to 10. For reference, a value of a health variable may be directly extracted from the health record, or may be derived by analyzing the record.

As another example, the internal integration server 25 may provide a notification to the user and/or the medical staff (e.g., medical staff in charge of treating the user) when a predicted risk level satisfies a preset condition (e.g., a threshold value or more). That is, a notification message may be transmitted to the user and/or the medical staff. The notification message may be transmitted to the corresponding user through, for example, the health management app installed in the user terminal 11, but the scope of the present disclosure is not limited thereto. The notification message may include, for example, a type of the health abnormality, a risk level, an expected occurrence time point, an occurrence cause, an expected duration, a recommended action, or a health management guideline, but is not limited thereto.

In the above example, a specific method of providing the notification may be designed in various ways.

For example, the internal integration server 25 may immediately provide the notification when the predicted risk level is the threshold value or more.

As another example, when the predicted risk level is the threshold value or more, the internal integration server 25 may predict a duration of the health abnormality through the machine learning model. Then, when the predicted duration is a reference value or more, the internal integration server 25 may immediately provide the notification. In an opposite case, the internal integration server 25 may withhold providing the notification.

As another example, when the predicted risk level is the threshold value or more, the internal integration server 25 may withhold providing the notification until a standby timer expires, and monitor a health-related event (e.g., a meal, exercise, a sleep, medication administration, etc.) of the user. In addition, when an event increasing a health risk (i.e., a risk of an occurrence of the health abnormality) is detected before the standby timer expires, the internal integration server 25 may immediately provide the notification. Conversely, when an event decreasing the health risk is detected, the internal integration server 25 may reset the standby timer and predict the risk level again through the machine learning model (i.e., perform the prediction again based on newly collected health variable data, etc.). Such a method may reduce user inconvenience caused by unnecessarily frequent notifications and also contribute to solving a problem in which a notification effect is degraded. For reference, even the same event may be determined as an event increasing the health risk or an event decreasing the health risk according to a state of the user (e.g., if the user is in a fasting state, the exercise may be determined as an event increasing the health risk). In addition, an expiration time of the standby timer may be dynamically set. For example, the expiration time may be dynamically set according to the predicted risk level, the expected occurrence time point, etc. for the health abnormality (e.g., when the predicted risk level is low and the expected occurrence time point is relatively far, the expiration time may be set to be longer).

As another example, when the predicted risk level is the threshold value or more, the internal integration server 25 may predict an occurrence time point of the health abnormality through the machine learning model. In addition, when a difference between a current time point and the predicted occurrence time point is less than a reference value, the internal integration server 25 may immediately provide the notification. If the difference is the reference value or more, the internal integration server 25 may withhold providing the notification until the standby timer expires, and monitor the health-related event (e.g., a meal, exercise, a sleep, medication administration, etc.) of the user. Then, when the event (e.g., exercise, medication administration, etc.) decreasing the health risk (i.e., the risk of the occurrence of the health abnormality) is detected, the internal integration server 25 may predict the risk level again through the machine learning model. Conversely, when the event increasing the health risk (e.g., a high-calorie meal, non-administration of medication at a set time, exercise on an empty stomach, etc.) is detected, the internal integration server 25 may immediately provide the notification.

For reference, the term ‘notification’ may be used interchangeably with expressions such as ‘alarm’, ‘alert’, ‘warning’, and the like depending on a context.

As described above, the operating method of the system 10 according to some embodiments of the present disclosure has been described with reference to FIGS. 3 to 5. According to the above description, since the system 10 performing health management interoperates with the legacy system 12 of the medical institution, a comprehensive and integrated health management service may be provided to the user. In addition, since the system 10 is configured to perform processing (e.g., analysis, monitoring, etc.) on health data independently of the legacy system 12, health-related information of the user may be reflected (or integrated) into the legacy system 12 without affecting a structure or performance of the legacy system 12.

In addition, since the system 10 provides the health-related information of the user to the medical staff through a dashboard, the integrated health management service may be more effectively provided to the user.

In addition, by comprehensively analyzing health variable data and clinical information of the user through a machine learning model, a risk level of an occurrence of the health abnormality for the user may be accurately predicted. In addition, when the risk level is a threshold value or more, the notification is provided to the user and/or the medical staff, whereby a situation in which the health abnormality occurs to the user may be prevented in advance.

Hereinafter, a design example of a dashboard 61 will be described with reference to FIGS. 6 to 7D.

FIG. 6 is an exemplary view illustrating a screen configuration of the dashboard 61.

As illustrated in FIG. 6, the dashboard 61 may include a plurality of interfaces 62 to 66 (or areas).

The first interface 62 is an interface for receiving an input of a search condition. The search condition may be, for example, a period, a user (patient) name, a hospital room number, and the like, but is not limited thereto. FIG. 7A illustrates implementation examples of the first interface 62 and the second interface 63, and exemplifies a case in which the first interface 62 is implemented to receive period information as the search condition (e.g., see a ‘period input part’). Information displayed on the interfaces 64 and 65 may be understood to be information meeting the search condition.

Next, the second interface 63 is an interface for displaying basic information (e.g., personal information, etc.) of the user. FIG. 7A exemplifies a case in which the second interface 63 is implemented to display a patient registration number, a user name, a date of birth, and the like.

Next, the third interface 64 is an interface for displaying health habit information. For example, the third interface 64 may be configured to display habit information (e.g., a time, a required time, the number of times/frequency, etc.) related to a sleep, a meal, exercise, drinking, and the like, and an evaluation score based thereon, and may be configured to visualize a change trend of the evaluation score. FIG. 7B exemplifies a case in which the third interface 64 is implemented to display daily health habit information, the change trend of the evaluation score over time (e.g., refer to a ‘graph’), and the like.

Next, the fourth interface 65 is an interface for displaying physical information. Here, the physical information may further include body composition analysis information and the like in addition to weight information submitted by the user. An implementation example of the fourth interface 65 will be described with reference to FIG. 7C. FIG. 7C exemplifies a case in which the fourth interface 65 is implemented to display daily weight information, daily body composition analysis information, a weight change graph, and the like.

In some cases, the dashboard 61 may further include a fifth interface (not shown) displaying an expected success rate for a health target (e.g., weight loss, visceral fat loss, etc.) set by the user and/or the medical staff. The fifth interface (not shown) may be configured to, for example, be displayed as a user input is received on a button object of the dashboard 61, but the scope of the present disclosure is not limited thereto. An implementation example of the fifth interface will be described with reference to FIG. 7D. FIG. 7D exemplifies a case in which the fifth interface is implemented to display a daily expected success rate, a change trend of the expected success rate over time (see the graph), and the like.

When the health target is the weight loss, the expected success rate may be calculated based on, for example, a weight change for 14 days, the number of times of health record submissions, whether the weight is lost, and the like, but is not limited thereto.

In some embodiments, the internal integration server 25 may accurately predict the success rate (i.e., the expected success rate) for the health target using the machine learning model, which will be described in detail below with reference to FIG. 11.

As described above, the design example of the dashboard 61 has been described with reference to FIGS. 6 to 7D. The above-described dashboard 61 may be utilized to effectively monitor and manage the health state of the user by visually providing various health-related information of the user.

Hereinafter, an exemplary structure, training method, and the like of the machine learning model 80 used for health abnormality prediction will be described with reference to FIGS. 8 to 10.

FIG. 8 illustrates input/output data of the machine learning model 80 according to some embodiments of the present disclosure.

As illustrated in FIG. 8, the machine learning model 80 may be configured to receive clinical information (or medical information), such as demographic information, diagnosis information, prescription information, a medication history, a disease history, and the like, and health variable data of a user as inputs, and make an early prediction of health abnormality information and output it. That is, the machine learning model 80 may be configured to predict information regarding a health abnormality by comprehensively analyzing the clinical information and the health variable data of the user. In this way, when even clinical information of an individual user is considered, prediction on health abnormality may be performed in a more precise and personalized form.

The health variable data may include values of various health variables, and the health variables may include, for example, a weight, a Body Mass Index (BMI), a waist circumference, a body fat percentage, a muscle mass, blood pressure, a heart rate, blood sugar, a sleep time, a meal time, the number of meals, an exercise time, the number of exercises, the number of drinking, the number of smoking, a stress index, or a combination thereof, but are not limited thereto.

The health abnormality information may include, for example, a type of the health abnormality (e.g., . . . ), whether the health abnormality occurs, an expected occurrence time point thereof, a duration thereof, an occurrence cause thereof, or a combination thereof, but is not limited thereto.

The machine learning model 80 may be designed and implemented in various ways. For example, the machine learning model 80 may be designed and implemented based on various types of models/algorithms such as a decision tree, a random forest, an extreme Gradient Boosting (XGBoost), a Light Gradient Boosting Machine (LightGBM), a Categorical Boosting (CatBoost), a Support Vector Machine (SVM), a logistic regression, an Adaptive Boost (AdaBoost), and a neural network, and a specific type/form/structure, etc. thereof may vary depending on a task of the machine learning model 80. Since those skilled in the art will already be familiar with a basic structure, a training method, and the like of the exemplified models, a description thereof will be omitted.

The above-described machine learning model 80 may be trained in a supervised learning method using a dataset of normal people and patients (i.e., people with a health abnormality). Since those skilled in the art will already be familiar with a meaning of the training being performed in the supervised learning method, a description thereof will be omitted. For reference, details of a label used for the supervised learning may vary depending on the task of the machine learning model 80.

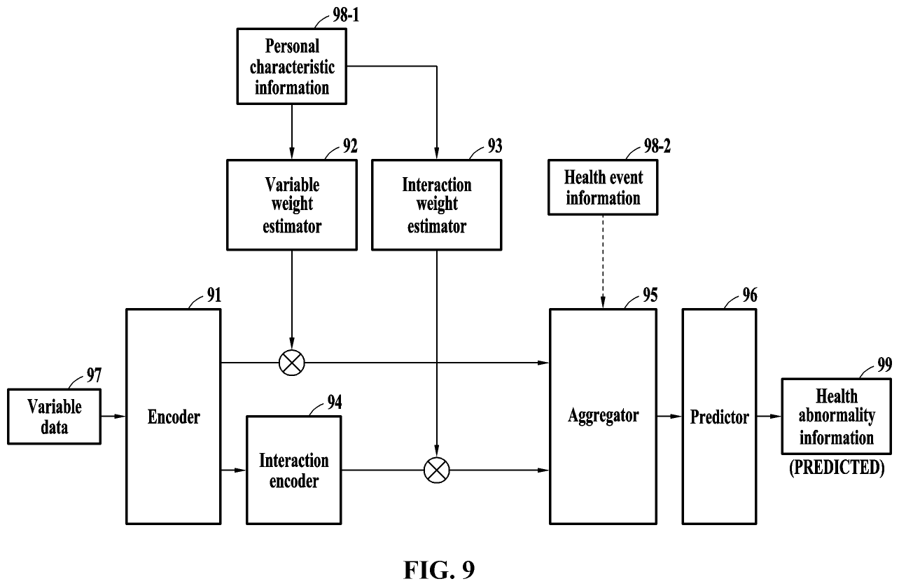

FIG. 9 is an exemplary view for explaining a structure and an operation of the machine learning model 80 according to some embodiments of the present disclosure. FIG. 9 illustrates a structure of the machine learning model 80 designed to perform a prediction specialized for an individual user, and assumes a case in which the model 80 is implemented based on a neural network.

As illustrated in FIG. 9, in some embodiments, the machine learning model 80 may include an encoder 91, a variable weight estimator 92, an interaction weight estimator 93, an interaction encoder 94, an aggregator 95, and a predictor 96.

The encoder 91 is a neural network module generating an embedding by encoding variable data 97. Here, the variable data 97 may be composed of values of various health variables, and may correspond to time-series data. For example, the encoder 91 may be configured to receive the value of each of the health variables and generate a variable embedding (e.g., an embedding vector) corresponding to each of them.

The encoder 91 may be implemented as any type/form of neural network. For example, the encoder 91 may be implemented based on a Multi-Layer Perceptron (MLP) (or a fully-connected layer), or may be implemented as another form of neural network.

Next, the variable weight estimator 92 is a module estimating a weight assigned to each of the health variables based on personal characteristic information 98-1 of the user. Here, the personal characteristic information 98-1 may include the clinical information of the user, and may further include other characteristic information. In addition, the personal characteristic information 98-1 may be encoded into an appropriate format and input to the variable weight estimator 92. For example, the variable weight estimator 92 may be configured to analyze the clinical information 98-1 of the user to estimate and output the weight of each of the health variables specialized for the individual user (i.e., a variable weight). Such a variable weight may be understood as a factor for reflecting the point that a degree of influence of each health variable on the health abnormality may vary for each user.

In some cases, the variable weight estimator 92 may be configured to receive a representative vector (e.g., a center vector, a weighted average vector, etc.) of a cluster to which the personal characteristic information 98-1 of the user belongs, and such technical contents may also be applied to the interaction weight estimator 93.

The variable weight estimator 92 may be implemented in any form. For example, the variable weight estimator 92 may be implemented based on a neural network module such as a Multi-Layer Perceptron (MLP), or may be implemented as a module performing an attention operation.

Next, the interaction weight estimator 93 is a module estimating an interaction weight assigned to predefined health variable pairs based on the personal characteristic information 98-1 of the user. For example, the interaction weight estimator 93 may be configured to analyze the clinical information 98-1 of the user to estimate and output a weight (i.e., an interaction weight) of each of the health variable pairs specialized for the individual user. Such an interaction weight may be understood as a factor for reflecting the point that a degree of influence of an interaction between the health variables on the health abnormality may vary for each user. The interaction weight estimator 93 may be implemented in a similar manner to the variable weight estimator 92.

Next, the interaction encoder 94 is a module generating an interaction embedding corresponding to each of the health variable pairs. For example, the interaction encoder 94 may be configured to generate an interaction embedding corresponding to each of the predefined health variable pairs based on the variable embeddings output from the encoder 91.

The interaction encoder 94 may be implemented in any form. For example, the interaction encoder 94 may be implemented based on a neural network module such as an MLP, or may be implemented as a module performing an operation such as an average, a concatenation, an elementwise multiplication, and the like on two variable embeddings.

Next, the aggregator 95 is a module generating an integrated embedding by aggregating the variable embeddings and the interaction embeddings to which weights are reflected. In other words, the aggregator 95 may be configured to generate the integrated embedding by aggregating the variable embeddings, to which the variable weight is reflected, and the interaction embeddings to which the interaction weight is reflected.

The aggregator 95 may be implemented in any form. For example, the aggregator 95 may be implemented based on a neural network module such as an MLP, or may be implemented in another way.

In some embodiments, the aggregator 95 may be configured to further receive health-related event information 98-2 of the user, as illustrated. The health-related event information 98-2 may be encoded in an appropriate manner and input to the aggregator 95. In this case, the predictor 96 may predict health abnormality information 99 by even considering a health-related event (e.g., exercise, sleep, a meal, drug administration, etc.) of the user.

Finally, the predictor 96 is a neural network module predicting the health abnormality information 99 based on the integrated embedding. That is, the predictor 96 may be configured to predict the health abnormality information 99 of the individual user by comprehensively analyzing various information (e.g., values of the health variables and various clinical information) contained in the integrated embedding. Such a predictor 96 may be implemented based on, for example, an MLP, but the scope of the present disclosure is not limited thereto.

For example, the predictor 96 may be configured to output a risk level (i.e., a probability) of an occurrence of the health abnormality to the user within (or after) a specific time/period (e.g., 6 hours, 12 hours, 1 day, 3 days, etc.) (e.g., configured to output a confidence score on whether the health abnormality occurs), or may be configured to output a type of the health abnormality, an occurrence cause thereof, an expected occurrence time point thereof, a duration thereof, and the like (e.g., configured to output a confidence score for each occurrence cause).

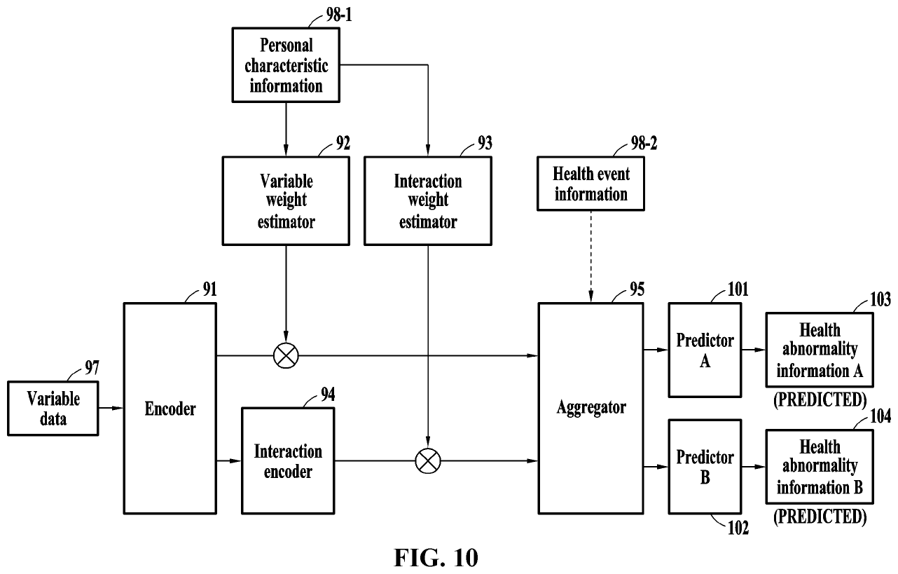

In some embodiments, the machine learning model 80 predicting the health abnormality information more accurately may be built through a multi-task learning technique. Hereinafter, such embodiments will be described with reference to FIG. 10.

FIG. 10 is an exemplary view for explaining a model training method based on multi-task learning according to some embodiments of the present disclosure. Hereinafter, for clarity of the present disclosure, names such as ‘predictor A’ and ‘predictor B’ will be named ‘a first predictor’ and ‘a second predictor’, and such a naming rule will be equally applied to other items in which similar names are used.

As shown in FIG. 10, the system 10 may perform the multi-task learning using a plurality of predictors (e.g., 101 and 102) connected to the machine learning model 80. FIG. 10 exemplifies a case in which two predictors 101 and 102 are connected to the aggregator 95 of the machine learning model 80, but the scope of the present disclosure is not limited thereto. For example, a third predictor (not shown) may be further connected to the aggregator 95. At least one of the predictors 101 and 102 may mean the predictor 96 of FIG. 9 performing a target task.

The first predictor 101 is a neural network module predicting first health abnormality information 103 (hereinafter, abbreviated as ‘first information’), and the second predictor 102 is a neural network module predicting second health abnormality information 104 (hereinafter, abbreviated as ‘second information’) different from the first information (for an example of each information, see below). That is, the first predictor 101 and the second predictor 102 are configured to perform different tasks (i.e., a task of predicting the first information and a task of predicting the second information) based on the integrated embedding.

The type and number of tasks for the multi-task learning may be variously defined and designed.

For example, the machine learning model 80 may be trained through a first task of predicting a risk level of an occurrence of a health abnormality within a specific time and a second task of predicting an occurrence cause of the health abnormality (that is, the first information 103 is the risk level of the occurrence of the health abnormality within the specific time, and the second information is the occurrence cause of the health abnormality). That is, parameters of the first predictor 101, the aggregator 95, the encoders 91 and 94, and/or the weight estimators 92 and 93 may be updated based on a prediction loss of the first task, and parameters of the second predictor 102, the aggregator 95, the encoders 91 and 94, and/or the weight estimators 92 and 93 may be updated based on a prediction loss of the second task.

As another example, the machine learning model 80 may be trained through a first task of predicting a risk level of an occurrence of the health abnormality within a first time and a second task of predicting a risk level of an occurrence of the health abnormality within a second time different from the first time (that is, the first information is the risk level of the occurrence of the health abnormality within the first time, and the second information is the risk level of the occurrence of the health abnormality within the second time). As a more specific example, the machine learning model 80 may be trained by performing a first task of predicting a risk level of an occurrence of the health abnormality within 24 hours through the first predictor 101, and performing a second task of predicting a risk level of an occurrence of the health abnormality within 48 hours through the second predictor 102.

As another example, the machine learning model 80 may be trained through a first task of predicting a risk level of an occurrence of the health abnormality within a specific time and a second task of predicting an occurrence time point of the health abnormality (that is, the first information is the risk level of the occurrence of the health abnormality within the specific time, and the second information is an expected occurrence time point of the health abnormality). As a more specific example, the machine learning model 80 may be trained by performing a first task of predicting a risk level of an occurrence of the health abnormality within 24 hours through the first predictor 101, and performing a second task of predicting a time point at which the health abnormality will occur through the second predictor 102 corresponding to a regression head.

As another example, the machine learning model 80 may be trained through a first task of predicting a risk level of an occurrence of the health abnormality within a specific time and a second task of predicting the number of occurrences (or a duration, etc.) of the health abnormality during a specific time (that is, the first information is the risk level of the occurrence of the health abnormality within the specific time, and the second information is the expected number of occurrences of the health abnormality during the specific time).

As another example, the machine learning model 80 may be trained using a task of predicting an accumulated health abnormality time during a specific time (or period), a task of predicting a value of a health variable after a specific time, a task of predicting a health-related event affecting health data, and the like.

As another example, the machine learning model 80 may be trained based on various combinations of the above-described examples.

When the training of the machine learning model 80 is completed, the system 10 may predict the health abnormality information of the user using this and provide a notification. At this time, the health abnormality information predicted through the machine learning model 80 may be used to determine whether to provide the notification, a notification method, a content of a notification message, and the like. For example, a risk level of an occurrence of the health abnormality, an expected occurrence time point thereof, a duration thereof, and the like may be used to determine whether to provide the notification, the notification method, and/or a health management guideline to be included in the notification message.

In some embodiments, the system 10 may derive a customized health management guideline for the user through the machine learning model 80. Specifically, when the risk level of the occurrence of the health abnormality is predicted to be a threshold value or more, the system 10 may repeatedly perform a risk level prediction while changing the health-related event information 98-2. Then, the system 10 may determine the customized health management guideline for the user based on the health-related event information 98-2 that has reduced the predicted risk level the most. For example, if the predicted risk level is reduced the most when the health-related event is ‘exercise’, the system 10 may determine the health management guideline of the corresponding user as ‘exercise’.

As described above, the input/output data, the exemplary detailed structure, the training method, and the like of the machine learning model 80 according to some embodiments of the present disclosure have been described with reference to FIGS. 8 to 10. According to the above description, by comprehensively analyzing the health variable data and individual characteristic information (e.g., clinical information) of the user through the machine learning model 80, the risk level of the occurrence of the health abnormality for the user may be accurately predicted. In addition, when the risk level is a threshold value or more, the notification is provided to the user and/or the medical staff, whereby a situation in which the health abnormality occurs in the user may be prevented in advance.

Hereinafter, a machine learning model 110 predicting a success rate for a health target will be described with reference to FIG. 11.

FIG. 11 is an exemplary view for explaining a structure and an operation of the machine learning model 110 according to some embodiments of the present disclosure.

As shown in FIG. 11, the machine learning model 110 may include a habit encoder 111, a willingness encoder 112, an environment encoder 113, an aggregator 114, and a predictor 115.

The habit encoder 111 is a neural network module generating a habit embedding by encoding health habit information 116 of the user. As described above, the health habit information 116 may include, for example, information (e.g., a time, a required time, a frequency, etc.) on a health habit (e.g., a habit related to exercise, a meal, a sleep, drinking, etc.) practiced (and/or not practiced) by the user, and may correspond to time-series data (e.g., daily health habit information, etc.).

The habit encoder 111 may be implemented as any type/form of neural network. For example, the habit encoder 111 may be implemented based on a Multi-Layer Perceptron (MLP), or may be implemented as another form of neural network (e.g., a Recurrent Neural Network (RNN) or a transformer-based neural network for encoding the time-series data).

Next, the willingness encoder 112 is a neural network module generating a willingness embedding by encoding willingness information 117 of the user. Here, the willingness information 117 is information directly or indirectly indicating the willingness of the user to achieve the health target, and may include, for example, a weight change, a voluntary submission frequency for the health habit information, a change frequency of the health target, a health target maintenance period (e.g., an average maintenance period), a target discontinuation rate, a self-motivation score input by the user, or a combination thereof, but is not limited thereto.

The willingness encoder 112 may be implemented, for example, in a similar manner/form to the habit encoder 111.

Next, the environment encoder 113 is a neural network module generating an environment embedding by encoding living environment information 118 of the user. The living environment information 118 is information on an external factor interfering with and/or helping achievement of a health target, and may include, for example, information on a sleep environment (e.g., a temperature, humidity, a noise, etc. of a residence), an exercise environment (e.g., a temperature, humidity, a distance to an exercise place, etc.), an external environment (e.g., weather, fine dust, etc.), a work environment (e.g., a work type, a work time, etc.), and a temptation environment (e.g., a density of restaurants, convenience stores, dessert cafes, etc. located near the residence or within an action radius), but is not limited thereto.

The environment encoder 113 may be implemented, for example, in a similar manner/form to the habit encoder 111.

Next, the aggregator 114 is a module generating an integrated embedding by aggregating the habit embedding, the willingness embedding, and the environment embedding. Here, the aggregator 114 may generate the integrated embedding by further aggregating health target information 119-1 set by the user and/or the medical staff. The health target information 119-1 may be encoded in an appropriate manner and input to the aggregator 114. The aggregator 114 will be described with reference to the description of the aggregator 95 of FIG. 9.

In some embodiments, the aggregator 114 may be configured to further receive an input of personal characteristic information (not shown) of the user. The personal characteristic information may be encoded in an appropriate manner and input to the aggregator 114. In this case, the predictor 115 may make a personalized prediction for a success rate 119-2 by further considering a characteristic of the user.

Finally, the predictor 115 is a neural network module predicting the success rate 119-2 for the health target of the user based on the integrated embedding. That is, a predictor 115 may be configured to predict the success rate 119-2 for the health target by comprehensively analyzing various information (e.g., information on the health habit, the willingness, the living environment, etc.) contained in the integrated embedding. Such a predictor 115 may be implemented, for example, based on an MLP, but the scope of the present disclosure is not limited thereto.

The above-described machine learning model 110 may be trained in a supervised learning method. That is, the machine learning model 110 may be trained using a success (or achievement) status of past users for the health target as label information.

As described above, the exemplary structure and operation of the machine learning model 110 for accurately predicting the success rate for the health target have been described with reference to FIG. 11. Hereinafter, an exemplary computing device 120 capable of implementing the above-described system 10 or components 21 to 25 thereof will be described with reference to FIG. 12.

FIG. 12 is an exemplary hardware configuration diagram illustrating the computing device 120.

As illustrated in FIG. 12, the computing device 120 may include at least one processor 121, a bus 123, a communication interface 124, a memory 122 loading a computer program 126 executed by the processor 121, and a storage 125 storing the computer program 126. However, FIG. 12 illustrates only components related to an embodiment of the present disclosure. Therefore, those skilled in the art to which the present disclosure pertains will appreciate that other general-purpose components may be further included in addition to the components illustrated in FIG. 12. That is, the computing device 120 may further include various components in addition to the components illustrated in FIG. 12. In some cases, the computing device 120 may be configured in a form in which some of the components illustrated in FIG. 12 are omitted. Hereinafter, each component of the computing device 120 will be described.

The processor 121 may control an overall operation of each component of the computing device 120. The processor 121 may include at least one of a Central Processing Unit (CPU), a Micro Processor Unit (MPU), a Micro Controller Unit (MCU), a Graphic Processing Unit (GPU), a Neural Processing Unit (NPU), a Tensor Processing Unit (TPU), a Vision Processing Unit (VPU), an Accelerated Processing Unit (APU), or any type of processor well known in the art of the present disclosure. In addition, the processor 121 may perform an operation on at least one application or program for executing specific operations/steps/methods. The computing device 120 may include one or more processors.

Next, the memory 122 may store various data, requests, and/or information. The memory 122 may load the computer program 126 from the storage 125 to execute specific operations/steps/methods. The memory 122 may be implemented as a volatile memory such as a RAM, but the technical scope of the present disclosure is not limited thereto.

Next, the bus 123 may provide a communication function between the components of the computing device 120. The bus 123 may be implemented as various types of buses such as an address bus, a data bus, a control bus, and the like.

Next, the communication interface 124 may support wired/wireless communication of the computing device 120. To this end, the communication interface 124 may include a communication module (e.g., an RF module supporting wireless communication) well known in the art of the present disclosure, and may support various communication methods.

Next, the storage 125 may non-temporarily store one or more computer programs 126. The storage 125 may include a non-volatile memory such as a Read Only Memory (ROM), an Erasable Programmable ROM (EPROM), an Electrically Erasable Programmable ROM (EEPROM) or a flash memory, a hard disk, a removable disk, or any type of computer-readable recording medium well known in the art to which the present disclosure pertains.

Next, the computer program 126 may include instructions causing the processor 121 to perform specific operations/steps/methods when loaded into the memory 122. That is, the processor 121 may perform specific operations/steps/methods by executing the loaded instructions.

For example, the computer program 126 may include instructions for an operation of acquiring health data of a user and an operation of providing the health data through a dashboard. In this case, the internal integration server 25 according to some embodiments of the present disclosure may be implemented through the computing device 120.

As another example, the computer program 126 may include instructions causing to perform at least a part of the operations/steps/methods described with reference to FIGS. 1 to 11. In the exemplified case, the system 10 and/or the components 21 to 25 thereof according to some embodiments of the present disclosure may be implemented through the computing device 120.

In some embodiments, the computing device 120 illustrated in FIG. 12 may mean a virtual machine implemented based on a cloud technology. For example, the computing device 120 may be a virtual machine operating in one or more physical servers included in a server farm. In this case, at least a part of the processor 121, the memory 122, and the storage 125 illustrated in FIG. 12 may be virtual hardware, and the communication interface 124 may also be implemented as a virtualized networking element such as a virtual switch or the like.

As described above, the exemplary computing device 120 capable of implementing the system 10 according to some embodiments of the present disclosure has been described with reference to FIG. 12.

As described above, various embodiments of the present disclosure and effects according to the embodiments have been mentioned with reference to FIGS. 1 to 12. Effects according to the technical spirit of the present disclosure are not limited to the effects mentioned above, and other effects not mentioned may be clearly understood by those skilled in the art from the following description.

According to some embodiments of the present disclosure, a health management system interoperates with a legacy system of a medical institution, so that a comprehensive and integrated health management service can be provided to a user. In addition, since the health management system is configured to perform processing (e.g., analysis, monitoring, etc.) on health data independently of the legacy system, health-related information of the user can be reflected (or integrated) into the legacy system without affecting a structure or performance of the legacy system.

In addition, since the health management system provides the health-related information of the user to medical staff through a dashboard, the integrated health management service can be more effectively provided to the user.

In addition, since the health management system includes a Backend For Frontend (BFF) server, a Fast Healthcare Interoperability Resource (FHIR) relay server, a gateway server, an FHIR server, and an internal integration server, and the FHIR server and the internal integration server among them are disposed in an internal network of the medical institution, interoperation between the health management system and the legacy system of the medical institution can be effectively performed.

Further, by comprehensively analyzing health variable data and clinical information of the user through a machine learning model, a risk level of an occurrence of a health abnormality for the user can be accurately predicted. In addition, when the risk level is a threshold value or more, a notification is provided to the user and/or the medical staff, whereby a situation in which the health abnormality occurs in the user can be prevented in advance.

Effects according to the technical spirit of the present disclosure are not limited to the effects mentioned above, and other effects not mentioned may be clearly understood by those skilled in the art from the following description.

In addition, even if it is described that a plurality of components are combined into one or operate in combination in the above embodiments, the technical spirit of the present disclosure is not necessarily limited to these embodiments. That is, within the scope of the purpose of the technical spirit of the present disclosure, all of the components may operate by being selectively combined into one or more.

The technical spirit of the present disclosure described above may be implemented as a computer-readable code on a computer-readable recording medium. The computer program recorded on the computer-readable recording medium may be transmitted to another computing device through a network such as the Internet and installed in the corresponding computing device, thereby being used in the corresponding computing device.

Although operations are shown in a specific order in the drawings, it should not be understood that the operations must necessarily be performed in the specific order shown in the drawings or in a sequential order, or that all the shown operations must be performed to obtain a desired result. In certain circumstances, multitasking and parallel processing may be advantageous. Although various embodiments of the present disclosure have been described with reference to the accompanying drawings, those of ordinary skill in the art to which the present disclosure pertains may understand that the technical spirit of the present disclosure may be implemented in other specific forms without changing the technical spirit or essential features thereof. Therefore, it should be understood that the embodiments described above are illustrative in all respects and not restrictive. The protection scope of the present disclosure should be construed by the following claims, and all technical spirits within the equivalent scope should be construed as being included in the scope of rights of the technical spirit defined by the present disclosure.

[Description of Symbols]

| | |
|---|---|
| 10: health management system | 11: User terminal |
| 12: legacy system | 21: BFF server |
| 22: FHIR relay server | 23: gateway server |
| 24: FHIR server | 25: internal integration server |
| 61: dashboard | 62, 63, 64, 656: interface |
| 80: machine learning model | 91: Encoder |
| 92: variable weight estimator | 93: interaction weight estimator |
| 94: interaction encoder | 95, 114: aggregator |
| 96, 101, 102, 115: predictor | 111: habit encoder |
| 112: willingness encoder | 113: environment encoder |

What is claimed is:

1. A system for health management, comprising:
a Backend For Frontend (BFF) server configured to perform an interface function with a health management app installed in a terminal of a user, and receive a service request from the health management app;
a Fast Healthcare Interoperability Resource (FHIR) relay server configured to generate an FHIR request by converting the service request according to an FHIR protocol, and transmit the FHIR request to an internal network of a medical institution; and
an FHIR server located in the internal network and configured to perform processing for the FHIR request,
wherein the system interoperates with a legacy system of the medical institution, and performs processing based on health data of the user independently of the legacy system.

2. The system according to claim 1, wherein the legacy system comprises an Electronic Medical Record (EMR) system.

3. The system according to claim 1, wherein the system provides at least a part of the health data to the legacy system, and is provided with medical information of the user from the legacy system.

4. The system according to claim 1, wherein the FHIR relay server transmits the FHIR request to the FHIR server through a gateway server that controls access to the internal network.

5. The system according to claim 1, wherein the service request comprises a verification request on whether the user is a registered patient in the medical institution,
the verification request comprises personal identification information of the user and is performed according to a membership registration application through the health management app,
the FHIR request comprises an FHIR verification request corresponding to the verification request, and
the FHIR server checks whether the user is a registered patient through the legacy system using the personal identification information, and returns an FHIR response comprising a verification result to the FHIR relay server.

6. The system according to claim 1, wherein the service request comprises an inquiry request for prescription information,
the inquiry request comprises patient identification information registered in the medical institution,
the FHIR request comprises an FHIR inquiry request corresponding to the inquiry request, and
the FHIR server acquires the prescription information through the legacy system using the patient identification information, and returns an FHIR response comprising the prescription information to the FHIR relay server.

7. The system according to claim 1, wherein the service request comprises a submission request for a health record,
the FHIR request comprises an FHIR submission request corresponding to the submission request, and
the FHIR server stores the health record in a database accessible by the legacy system, and returns an FHIR response comprising the storage result to the FHIR relay server.

8. The system according to claim 1, further comprising an internal integration server providing the health data by visualizing the health data through a dashboard,
wherein the health data comprises body composition analysis information of the user and a health record submitted by the user through the health management app,
wherein the body composition analysis information is acquired from a body composition analysis server comprised in the legacy system, and
the dashboard is configured to be accessible through a User Interface (UI) of an Electronic Medical Record (EMR) system comprised in the legacy system.

9. The system according to claim 8, wherein the health record comprises health habit information and weight information, and
the dashboard comprises: a first interface displaying basic information of the user; a second interface displaying the health habit information; and a third interface displaying physical information comprising the weight information and the body composition analysis information,
wherein the second interface further displays an evaluation score calculated based on the health habit information.

10. The system according to claim 8, wherein the internal integration server predicts a success rate for a health target of the user by analyzing the health record, and further provides the predicted success rate through the dashboard.

11. The system according to claim 10, wherein the internal integration server predicts the success rate through a machine learning model,
the health record comprises health habit information and weight information, and
the machine learning model comprises:
a habit encoder configured to generate a habit embedding by encoding the health habit information;
a willingness encoder configured to generate a willingness embedding by encoding willingness information of the user, the willingness information comprising at least one of a weight change of the user, a voluntary submission frequency for the health habit information, and a change frequency of the health target;
an environment encoder configured to generate an environment embedding by encoding living environment information of the user;
an aggregator configured to generate an integrated embedding by aggregating the habit embedding, the willingness embedding, and the environment embedding; and
a predictor configured to predict the success rate based on the integrated embedding.

12. The system according to claim 1, further comprising an internal integration server configured to predict a risk level of an occurrence of a health abnormality for the user by analyzing the health data through a machine learning model,
wherein the machine learning model comprises:
an encoder configured to generate variable embeddings corresponding to predefined health variables;
an interaction encoder configured to generate interaction embeddings corresponding to predefined health variable pairs based on the variable embeddings;
a variable weight estimator configured to estimate a variable weight specialized for the user with respect to each of the health variables based on clinical information of the user acquired from the legacy system;
an interaction weight estimator configured to estimate an interaction weight specialized for the user with respect to each of the health variable pairs based on the clinical information;
an aggregator configured to generate an integrated embedding by aggregating the variable embeddings, to which the variable weight is reflected, and the interaction embeddings to which the interaction weight is reflected; and
a predictor configured to predict the risk level based on the integrated embedding.

13. The system according to claim 12, wherein the internal integration server:
provides a notification related to the health abnormality when the predicted risk level is a threshold value or more and a preset standby timer expires,
immediately provides the notification when an event increasing a risk of the occurrence of the health abnormality for the user is detected before the standby timer expires, and
resets the standby timer and predicts the risk level again through the machine learning model when an event decreasing the risk of the occurrence of the health abnormality for the user is detected before the standby timer expires.
